(19) United States
(12) Patent Application Publication
(10) Pub. No.: US 2018/0064406 A1
(43) Pub. Date: Mar. 8, 2018

(54) SYSTEM FOR ASSISTING IN THE POSITIONING OF A INTRAORAL DENTAL RADIOLOGY SENSOR

(71) Applicant: Teledyne e2v Semiconductors SAS, Saint Egrève (FR)

(72) Inventors: Nathalie PASCAL, Le Sappey En Chartreuse (FR); Florian JULIEN, Mid Levels (HK)

(21) Appl. No.: 15/559,786

(22) PCT Filed: Feb. 26, 2016
(86) PCT No.: PCT/EP2016/054142

§ 371 Date: Sep. 19, 2017

(30) Foreign Application Priority Data
Mar. 20, 2015 (FR) 1552344

(51) Int. Cl. A61B 6/14; A61B 6/00; A61B 34/20

(52) U.S. Cl. CPC A61B 6/145; A61B 6/589; A61B 6/46; A61B 6/4452; A61B 6/588; A61B 34/20

(57) ABSTRACT

The invention relates to medical imaging and more particularly to intra-oral dental radiology. It proposes a radiological imaging assembly comprising an intra-oral image sensor 10, in which the sensor has an active face A corresponding to the photosensitive surface, and a mirror M1 which is placed in front of the active face of the sensor, the mirror face reflecting the visible light being turned in the same direction as the active face of the sensor, toward an X-ray source. The mirror can be disposed on the sensor itself, on the front face cap 23 of the encapsulation housing 20 of the sensor and/or on the outer face of an accessory containing or supporting the sensor, the outer face being turned in the same direction as the active face of the sensor.

### FIELD OF THE INVENTION

The invention relates to medical imaging and more particularly to intra-oral dental radiology. Dental radiology systems use an image sensor which is placed in mouth of the patient, behind and in proximity to the dental region to be observed. An X-ray source is placed outside the mouth of the patient, facing the sensor, and exposes the latter to a short flash of X-rays, through the biological tissues or other materials to be observed which are situated between the source and the active face of the sensor. The active face of the sensor is the face which is sensitive to the radiations, and which makes it possible to obtain the radiological snapshot of the anatomical zone that is to be observed.

### DESCRIPTION OF THE PRIOR ART

Among the major constraints in using dental radiological systems, it is most particularly essential to consider the risk of exposure of the patient and of his or her surroundings to the X-rays. It is necessary to minimize the dose of X-rays sent, while ensuring that a good image of the observed region is obtained.

Obtaining the good image presupposes that the sensor is placed correctly in the mouth of the patient, in relation to the anatomical zone that is to be X-rayed, failing which a part of the dose sent would be wasted.

However, correctly placing the sensor in the mouth of a patient is an operation which can prove difficult in practice, because of the darkness in the buccal cavity, its limited volume and the more or less easy access (mouth aperture diameter variable according to the patients).

For all its reasons, the practitioner may have difficulties in correctly positioning the active face of the sensor in relation to the anatomical part of the patient that he or she wants to X-ray (teeth, roots, etc.) and in visually checking, directly, and if necessary correcting, this positioning. The checking will therefore be done indirectly, on the snapshot obtained: if the positioning is not good, the snapshot obtained will be of no use to the practitioner, who will have to take a new X-ray snapshot.

Now, efforts are made to minimize the exposure of the people to the radiations as far as it is reasonably possible.

Support accessories are proposed to the practitioners to facilitate the correct placement of the image sensor and the holding in the correct position for the time it takes to perform the X-ray.

Some of these support accessories are specially designed to produce very precise snapshots. These are for example accessories including a sensor support zone and a zone to be etched.

Other support accessories allow all types of snapshots. These accessories generally comprise a sensor support zone at the end of a guiding and holding rod which facilitates the handling by the practitioner.

These support accessories do admittedly facilitate the placement and handling, but they are not sufficient to ensure, each time, a correct positioning of the sensor, quite simply because the mouth and dentition of each patient are unique. Each time, there is a real adjustment to be made. Also, the practitioner has great difficulty in being able to perform and check this adjustment, because of the darkness of the buccal cavity.

### SUMMARY OF THE INVENTION

One object of the invention is thus to assist in the positioning of the intra-oral dental sensor.

The invention proposes a radiological imaging assembly comprising an intra-oral radiological image sensor, the sensor comprising an active face and a rear face, the active face being intended to be turned toward an X-ray source.

According to the invention, this assembly comprises at least one mirror which is placed in front of the active face of the sensor, the mirror forming a face reflecting the visible light which is turned on the same side as the active face of the sensor.

In one embodiment, a mirror is formed on an outer face of an encapsulation housing enclosing the sensor, in which the outer face corresponds to the active face of the sensor. The mirror can have been formed previously on the front face cap of the housing, before encapsulation and welding of the cap with a rear face shell of the housing.

According to the invention, the imaging assembly can comprise at least one accessory supporting or containing the sensor in which an outer face of the accessory is placed in front of the active face of the sensor and turned in the same direction as the active face of the sensor, and a mirror formed on this outer face of the accessory.

The accessory can be a tool comprising a support zone secured to a rod, in which the support zone is designed to hold the sensor, with the active face of the sensor against the inner face of the support zone. The mirror can then be formed on an outer face of the support zone.

The accessory can be a hygienic protection enclosure, disposable or not, designed to enclose an intra-oral sensor. The mirror can then be formed on the outer surface of the enclosure.

The accessory can be a comfort flexible shell inside which the sensor is placed, with active face against a bottom wall of the shell, and a mirror is formed on the outer face of this wall.

The imaging assembly can comprise one or more of these accessories, and at least the outer(most) face of the imaging assembly, which is that of one of the accessories, comprises a mirror according to the invention.

The mirror can be formed by layer deposition of a reflecting material, by vacuum evaporation, on a front face cap of the housing before encapsulation, and/or on the front face of the encapsulation housing enclosing the sensor and/or on the outer face of the comfort shell and/or of the support zone of an accessory.

The mirror can also be formed by a mirror piece cut from a strip of material having a reflecting face and incorporated by molding in a thermoplastic material.

The mirror can also be formed by cutting and gluing a piece of mirror in a strip of material having a reflecting face and an adhesive other face.

### BRIEF DESCRIPTION OF THE DRAWINGS

Other features and advantages of the invention will become apparent on reading the following detailed description which is given with reference to the attached drawings in which:

FIG. 1 illustrates the principle of dental radiological imaging;

FIG. 2 is a front face view of an intra-oral dental radiological sensor;

FIG. 3 is a rear face view of an intra-oral dental radiological sensor;

FIG. 4 is a cross-sectional view of this sensor, showing the mounting of the electronic chip of the sensor, encapsulated in a housing;

FIG. 5 illustrates an example of a positioning-assisting mirror according to the invention formed on the front face of a radiological sensor;

FIG. 6 illustrates another example of a positioning-assisting mirror according to the invention formed on an outer face of an accessory support of a radiological sensor;

FIGS. 7 and 8 represent another example of a positioning-assisting mirror according to the invention formed on an outer face of a hygienic protection accessory of a radiological sensor, which is an enclosure in which the sensor is inserted and enclosed;

FIG. 9 represents a removable radiological sensor comfort shell; and

FIG. 10 represents another example of a positioning-assisting mirror according to the invention formed on an outer face of such a comfort shell; and

FIG. 11 illustrates a precut piece of reflecting material, for producing a positioning-assisting mirror according to the invention.

### DETAILED DESCRIPTION OF THE INVENTION

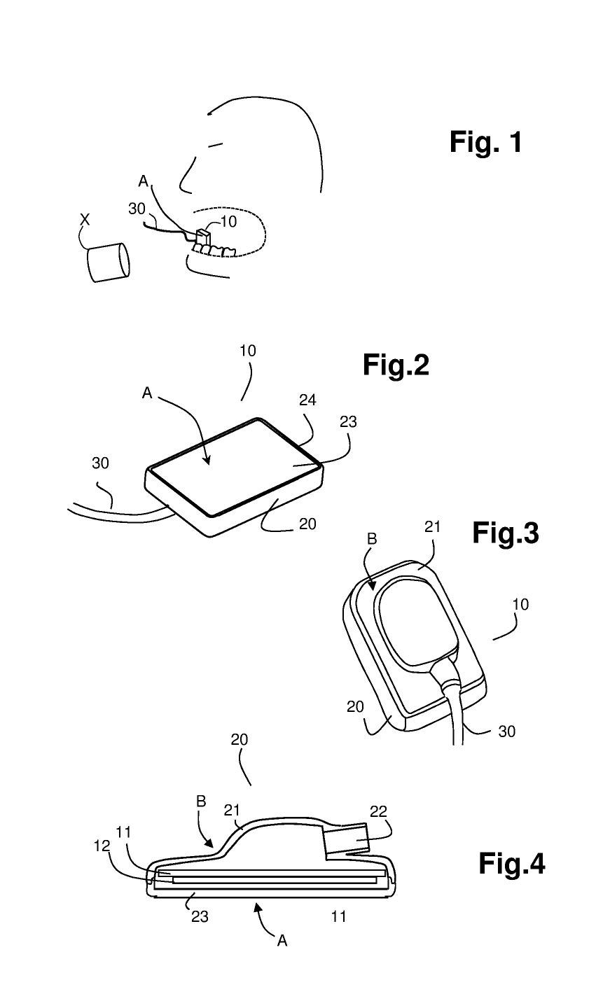

FIG. 1 gives a simplified representation of the principle and the elements of dental radiological imaging: an intra-oral dental radiological sensor 10 is placed in the mouth of a patient, its active face A behind the anatomical zone to be X-rayed. An external X-ray source is placed outside the mouth, in alignment with the zone to be X-rayed and the active face A of the sensor.

In the example, the sensor comprises an output cable 30 intended to be linked to a computer. The cable will allow the transmission of images and other possible interface or command signals and its electrical power supply or the recharging of a battery incorporated in the sensor. However, a wireless interface is possible.

A sensor should be understood to mean the assembly comprising a scintillator, an electronic circuit which performs the image capture and image transmission, and a protective housing in which this circuit is encapsulated. The electronic circuit can be of any prior art technology (CCD, MOS). As illustrated in the cross-sectional view of FIG. 4, the assembly comprising the electronic circuit 11 and the scintillator 12 is placed (possibly with other elements not represented such as stiffening elements, discrete components, a battery, etc.) in a shell 21 whose bottom forms the rear face B (FIG. 3) of the housing, with an aperture 22 provided to allow the passage of an output electrical cable 30. A front face cap 23 (FIG. 2) is added on top of the circuit and assembled with the shell, typically by ultrasound welding. The front face cap 23 corresponds to the active face A of the sensor: under the cap 23 there will thus be the scintillator 12, and under the scintillator, the matrix of photosensitive elements of the electronic circuit 11. The X radiations emitted by a source placed in front of this active face are transformed by the scintillator into light photons that can be detected by the photosensitive elements of the matrix.

The shell 21 and the front face cap 23 which form the housing 20 are designed in such a way as to provide the rigidity needed to protect the electronic circuit and in particular the photosensitive matrix, to allow a tight output with the output cable 30 and to also ensure in-mouth comfort for the patient. In particular: the housing, of rectangular form, can have cut corners and rounded edges; the front face cap can be made of hard plastic material; the edges of the housing can be made of flexible plastic with smooth gum appearance.

To assist the practitioner in correctly positioning the sensor in the mouth of a patient, the invention proposes placing at least one mirror in front of the active face of the sensor, with the reflecting surface of the mirror turned in the same direction as the active face A, that is to say toward the zone to be X-rayed and toward the external X-ray source (FIG. 1).

More specifically, according to the invention, a radiological imaging assembly comprising an intra-oral image sensor comprises a mirror disposed on an outer face of the imaging assembly, with a reflecting face of the mirror oriented on the same side as the active face A of the sensor.

The radiological imaging assembly (or a part thereof, obviously including the image sensor) is placed in the mouth of the patient, behind the anatomical zone to be X-rayed: on the surface of this mirror, illuminated by the visible light (natural, or, more efficiently, the light emitted by a lighting apparatus oriented toward the mouth of the patient), will thus be formed an image of the anatomical zone behind which the sensor is placed: the practitioner will be able to use this image to assist in adjusting the positioning of the sensor in relation to the zone that he or she wants to X-ray. The mirror will in practice be as transparent as possible to the X-rays so as not to affect the quality of the radiological image.

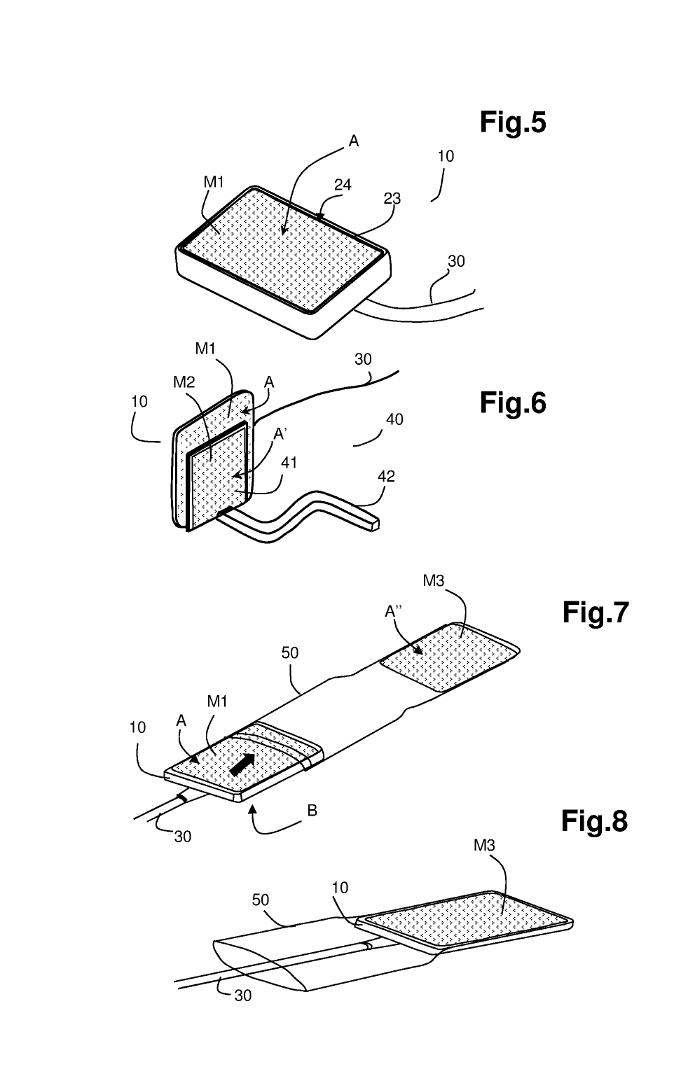

In one implementation of the invention, a positioning-assisting mirror according to the invention is incorporated in the sensor itself, as illustrated in FIG. 5: a mirror M1 is formed on the outer front face of the encapsulation housing 20 of the sensor corresponding to the active face A of the sensor: that is to say, to return to the example of housing illustrated in FIGS. 2 to 4, on the front face cap 23 of this housing, over all the surface of this cap apart from the border 24. In this embodiment, the surface of the mirror M1 is substantially equal and exactly positioned, by superpositioning, in the axis of the X-ray source on the photosensitive matrix surface of the sensor: the visible image which is formed on the mirror is close to the X-ray image which will be obtained, and the practitioner can perform the positioning of the sensor with precision by observing this image.

The mirror M1 can be formed before or after encapsulation of the sensor in its housing 20. If it is performed after encapsulation, it will be possible to use a technique of deposition of a reflecting material by vapor phase evaporation, with a masking tool to delimit the deposition zone and deposit the reflecting material over all the inner face of the cap 23, apart from the border 24.

It is also possible to produce the mirror before encapsulation. More specifically, provision can be made to produce the mirror M1 at the time of the fabrication of the front face cap 23, before encapsulation of the circuit and assembly with the rear face shell 21. In this variant, the same evaporation deposition technique can be used. However, it would also be possible to precut a piece P of mirror (FIG. 11), from a strip of material having a reflecting face, and press this piece P into the bottom of a mold, to mold, on top of and around this piece, the front face cap form. The piece of mirror is then incorporated in the cap, on the surface of this cap. The piece P of mirror could be used as bottom of the cap and form the border 24 by overmolding thermoplastic material.

It is also possible to provide for adding the piece P of mirror onto the front face cap of the housing by gluing. Preferably, provision can then be made to provide the piece P of mirror with an adhesive face, using a pressure-sensitive adhesive, which is protected by a peelable film. The mirror can then be placed by the practitioner on the sensor, that is to say on the front face of the encapsulation housing.

The imaging assemblies used by the practitioners can also comprise one or more accessories in addition to the sensor. There are accessories provided to assist in the handling and the placement of the sensor in the mouth of the patient, hygiene accessories, or even accessories provided to improve the comfort of the patients: whatever the accessory or accessories used, a mirror will preferably be formed on the outer surface of the imaging assembly, that is to say that which comes into the foreground, seen from the side of the practitioner looking toward the interior of the mouth of the patient, whether this is a face of the housing of the sensor or that of an accessory which is placed in the mouth between the zone to be X-rayed and the active face of the sensor.

These accessories are produced in materials suitable for medical or dental use (single-use or not) and transparent to X radiations, and in particular plastic materials, which are generally not transparent to visible light, and which, for this reason, can mask or render less visible (for the practitioner), at least partly, the active face of the sensor.

A first accessory can be a sensor support element 40 (“handler”) as illustrated in FIG. 6. The sensor support element comprises a sensor support 41, provided to place therein and hold a sensor 10, and a rod 42 secured to the support zone 41, that the practitioner will be able to handle. The rod may or may not be articulated. It may also be coupled to other elements (not represented), such as, for example, an X-ray source alignment window, or an element to be etched.

The support 41 comprises an outer face A′, on the rod side, and an inner face provided to receive the sensor, with the active face A of the sensor 10 against this inner face and to hold the sensor in this position: to this end, the inner face can include mechanical elements that are not represented, such as slideways or clamps for example, or else this inner face is an adhesive face, with an adhesive of pressure-sensitive type (PSA).

The support 41 takes the form, as illustrated in FIG. 6, of a substantially planar surface which at least partly masks the active face A of the sensor. In the example illustrated, the surface is solid. However, it could include one or more apertures.

According to the invention, a mirror M2 can be provided on the outer face A′ of the support 41, with reflecting face turned on the same side as the active face A of the sensor, that is to say toward the zone to be X-rayed. If, as represented, the sensor comprises a mirror M1 on its active face A, the two mirrors M1 and M2 are oriented on the same side, that is to say to face the external X-ray source (FIG. 1).

The mirror M2 can be produced on the face A′ of the support 41 by using the same techniques indicated above: vacuum evaporation; or else, when at least the support 41 is produced by molding a thermoplastic material, surface integration of this support by precutting of a piece of mirror and molding of the support form (or support element 40) on top of and around this piece; or even, gluing of a piece P of mirror.

When the support 41 is conformed to correspond to the exact dimensions of the sensor, for example in the case of a custom-made accessory, the dimensions of the mirror M2 will be advantageously able to correspond to the dimensions of the photosensitive matrix of the sensor. In this case, the mirror M2 formed on the support 41 allows the most accurate positioning of the sensor, because the image which will be formed on this mirror will correspond as closely as possible to the radiological image that the sensor will be able to detect. In this situation, the housing of the sensor does not need to be coated with a mirror. The same applies if the support 41 is larger than the sensor.

In the case where the support element is generic, the dimensions of the mirror produced on the outer face A′ of the sensor support will depend in practice on the surface of this support. In all cases, the aim will be to favor the accuracy of the positioning: that is to say that the aim will be to best match the size of the mirror with the size of the radiological image that the sensor can detect. For example, if the support is large enough, the mirror will preferably be dimensioned to correspond to sensors of average dimensions: that is to say neither the smallest nor the largest. In this way, not too much accuracy will be lost at the ends of the range. In another example, if the size of the support corresponds to the size of the smallest sensors, or is smaller, provision will be made for the mirror M2 to entirely cover the surface of the support, to offer the practitioner the image that is the closest possible to that supplied by the sensor. In this case, it may be preferable for the sensor to also be coated with a mirror as is represented in FIG. 6.

A second accessory can be a hygienic enclosure 50, generally for single use, as represented in FIGS. 7 and 8. Such an enclosure is used by the practitioner to improve the rules of cleanliness and hygiene. The enclosure comprises an aperture through which the sensor is introduced (FIG. 7) and pushed to the bottom of the enclosure. After the radiological snapshot has been taken, the imaging assembly is removed from the mouth of the patient, the sensor is removed from the enclosure and the enclosure is sterilized, or it is disposed of (single-use enclosure).

The hygienic enclosure is produced in a plastic material suitable for medical and dental use, transparent to the radiations and possibly transparent to visible light. However, even if it is in fact transparent to visible light, this transparency is not optimal, because its surface is not very reflective and, assuming that the sensor that it encloses comprises a mirror M1 according to the invention, on the active face of the sensor, the image formed on this mirror M1 will not be well-defined.

According to the invention, a mirror M3 can be provided on an outer face A″ of the enclosure 50, in the zone corresponding to the bottom of the enclosure.

The sensor must then be introduced with its active face A on the same side as the mirror M3 on the face A″ of the enclosure. If, as represented, the sensor comprises a mirror M1 on its active face A, the two mirrors M1 and M2 are then oriented on the same side, such that, once placed in the mouth of the patient, both will be oriented in the same direction, behind the anatomical zone to be X-rayed and facing the eternal X-ray source (FIG. 1). It is then the mirror M3 which is on the outer face of the duly constituted imaging assembly, which will allow the practitioner to check and adjust the positioning of the sensor in the mouth of the patient, by observation of the visible image which is formed on this mirror.

This mirror M3 will for example be formed by a vacuum evaporation technique, or else by gluing a piece of mirror P as detailed previously.

If the enclosure is specific to a type of sensor, that is to say adjusted precisely to the dimensions of the sensor, the mirror M3 will be produced on an outer face of this enclosure so as to cover a surface equivalent to the active face of the sensor, that is to say the inner surface of the front face cap apart from the border 24.

If the enclosure is generic, to be used with all the range of dimensions of the market-standard sensors: the mirror will preferably be dimensioned to correspond to sensors of average sizes, that is to say neither the smallest nor the largest. In this way, not too much accuracy will be lost at the ends of the range.

It will be noted that the imaging assembly can comprise, at the same time, a support accessory such as the element 40 of FIG. 6, a hygienic enclosure 50 and a sensor 10. In particular, it is possible to have a support element 40, on which will be positioned, by means of an adhesive provided on the inner face of the support 41, the part A″ in line with the active face A of the sensor contained in an enclosure. After the snapshot, the assembly is removed from the mouth of the patient, the sensor is extracted from the enclosure and the support element and/or the enclosure can be disposed of or sterilized for a new use.

In this case, according to the invention, the duly constituted imaging assembly, that is to say the support element 40 with its support 41 on which is disposed the sensor 10 enclosed in an enclosure 50, comprises at least one mirror (M2) on the outer face of the support 41: this mirror which is on the outer face of this imaging assembly will allow the practitioner to check and adjust the positioning of the sensor, by direct observation of the image which is formed on top.

However, the sensor and/or the enclosure of this assembly can also comprise a respective mirror (M1, M3) according to the invention.

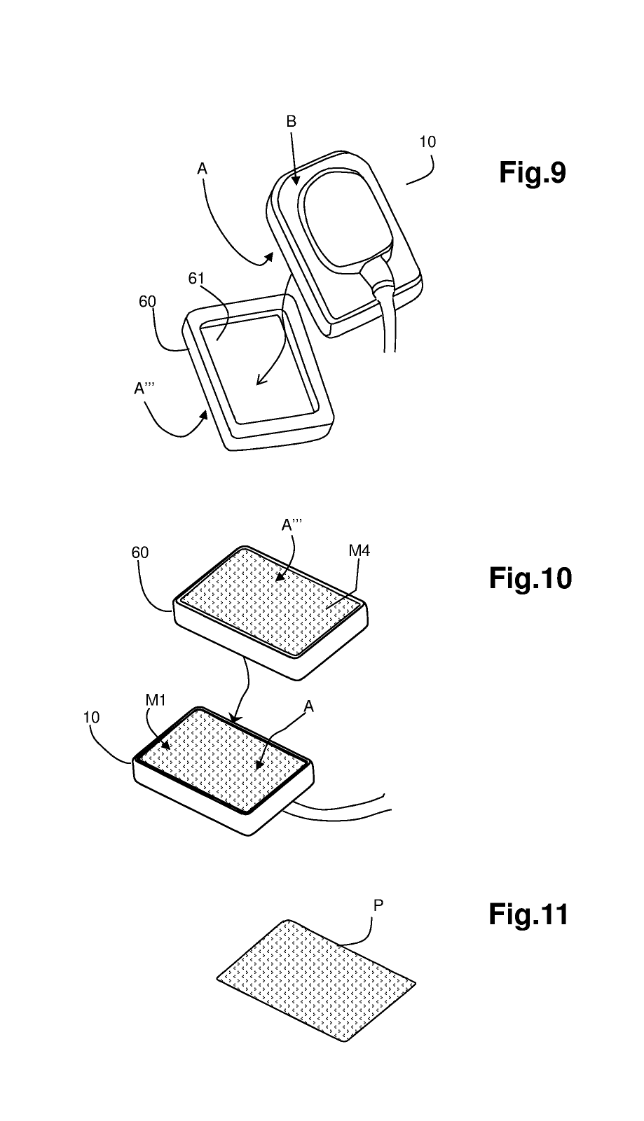

Another accessory which can be used by the practitioner is a flexible comfort shell 60 as schematically illustrated in FIGS. 9 and 10. These comfort shells are for example produced in a flexible plastic material with a smooth gum appearance. They make it possible to render the contact with the sensor more agreeable (when this sensor is in the mouth) and at the same time they dampen the effects of clamping or of any impacts between or against the teeth of the patient.

The practitioner uses the comfort shell as follows: he or she places the sensor 10 inside the shell, with the active face A of the sensor 10 pressed against the bottom wall 61 of the shell 60.

According to the invention and as represented in FIG. 10, it is possible to provide a mirror M4 on the outer face A′″ of the shell bottom wall 61, oriented on the same side as the active face A of the sensor, behind the anatomical zone to be X-rayed and facing the external X-ray source.

Thus, when an imaging assembly, consisting of a comfort shell comprising a mirror M4 on the outer face and a sensor 10 placed in this shell, is placed in the mouth of the patient, the mirror M4 will allow the practitioner to check and adjust the positioning of the sensor in the mouth of the patient, by observation of the visible image which is formed on this mirror. The sensor 10 does not need to be coated with a mirror, but it can be.

In the case where the sensor is coated with a mirror M1 on its active face A as represented in FIG. 10, the two mirrors M1 and M4 are oriented on the same side, toward the zone to be X-rayed.

The invention applies to an imaging assembly which can comprise just one sensor, or else a sensor in combination with one or a plurality of accessories supporting or containing this sensor. According to the invention, at least the outer face of this assembly is coated with a mirror such that, when the imaging assembly (or a part of this assembly including the sensor) is placed in the mouth behind the zone to be X-rayed, the reflecting face of this mirror is placed behind the zone to be X-rayed and in front of the active face A of the sensor.

If the imaging assembly consists only of the sensor: this mirror is the mirror M1 produced on the active face of the sensor.

If the imaging assembly consists of a combination of a sensor with at least one accessory: at least one mirror is produced on the outer face of the accessory which will be located just behind the zone to be X-rayed; and the sensor and/or the other accessory or accessories will each be able to be coated or not with a mirror.

For example, if an imaging assembly is envisaged that is formed by a support accessory 40 for a comfort shell 60 containing a sensor 10, this assembly comprises at least one mirror M2 on the outer face of the support zone of the shell. It may be advantageous to provide for the comfort shell 60 to be also coated with a mirror such as M4, particularly if the size of the support 41 is smaller than the shell. In this case, the reflecting face of this mirror M4 will be oriented like that of the mirror M2 and like the active face of the sensor, that is to say toward the zone to be X-rayed. In this example, it is not necessary for the housing of the sensor to be coated with a mirror.

Preferably, an assembly will be proposed to the practitioner comprising both a sensor whose housing comprises a reflecting mirror face on the side of its active face, one or more support elements whose support front face comprises a reflecting mirror face, and enclosures having a reflecting part. In this way, however the elements of the assembly are used, there is an assurance that there will always be a mirror face in immediate proximity to the zone to be X-rayed.

In practice, the mirror or mirrors will be formed from a material transparent to X radiations and compatible for medical or dental use, and which offers good quality adhesion to the plastic materials and in particular to the thermoplastic materials generally used for the shells, housings, support element, and to the plastic materials used to produce the hygienic enclosures. The mirror or mirrors will for example be formed from a metallic material such as aluminum, chromium or nickel for example, by using the usual deposition technologies, in particular vacuum evaporation, allowing depositions in thin layers of uniform thickness, such that the integration of the mirror or mirrors into the imaging assembly does not or at least does not significantly degrade the quality of the radiological image obtained.

1. A radiological imaging assembly comprising an intra-oral image sensor comprising an active face corresponding to the photosensitive surface, which is intended to be turned toward an X-ray source, and at least one mirror which is placed in front of the active face of the sensor, the mirror face reflecting the visible light being turned in the same direction as the active face of the sensor.

2. The imaging assembly of claim 1, in which a mirror is formed on a protective housing enclosing the sensor.

3. The imaging assembly of claim 2, in which said mirror is formed on a front face cap of the protective housing, before encapsulation and welding of the cap with a rear face shell of the housing.

4. The imaging assembly of claim 1, comprising at least one accessory supporting or containing the sensor and an outer face of the accessory is placed in front of the active face of the sensor and turned in the same direction as the active face of the sensor, and a mirror is formed on said outer face of the accessory.

5. The imaging assembly of claim 4, wherein the accessory comprises a support secured to a rod, the support having a substantially planar front face opposite a rear face, the sensor being placed on the support, with the active face of the sensor against the rear face of the support, and a mirror is formed on the front face of the support.

6. The imaging assembly of claim 4, wherein the accessory is a hygienic protection enclosure enclosing the sensor, and the enclosure comprises a mirror formed on an outer face of the enclosure, the sensor being placed inside the enclosure with its active face oriented on the same side as said outer face of the enclosure and under said mirror of the enclosure.

7. The imaging of claim 4, wherein the accessory is a comfort flexible shell inside which the sensor is placed, with the active face of said sensor against a bottom wall of the shell, and a mirror is formed on an outer face of said bottom wall of the shell.

8. The imaging assembly of claim 1, in which the at least one mirror is a piece cut from a strip of material having a reflecting face, incorporated by surface molding of a thermoplastic material.

9. The imaging assembly as claimed in claim 1, in which the at least one mirror is formed by deposition of a reflecting material by vacuum evaporation.

10. The imaging assembly of claim 1, in which the at least one mirror is a piece cut from a strip of material having a reflecting face and an adhesive other face.
